(19) United States
(12) Patent Application Publication
(10) Pub. No.: US 2005/0043682 A1
(43) Pub. Date: Feb. 24, 2005

(54) Flexible inflow/outflow cannula and flexible instrument port

(75) Inventors: Theodore R. Kucklick, Los Gatos, CA (US); Robert P. Bruce, Los Gatos, CA (US)

Correspondence Address: Crockett & Crockett, Suite 400, 24012 Calle De La Plata, Laguna Hills, CA, 92653, US

(73) Assignee: Cannuflow Incorporated

(21) Appl. No.: 10/646,442

(22) Filed: Aug. 22, 2003

(51) Int. Cl. A61M 5/178

(52) U.S. Cl. 604/164.09; 606/108; 604/167.03

(57) ABSTRACT

A flexible plastic cannula for use during arthroscopic surgery. The cannula is a flexible elongated tube having longitudinally staggered slots disposed on the distal end of the tube. The tube is tapered along its length so that the proximal portion of the tube is relatively rigid, while the distal end is narrower and more flexible. The tube is sufficiently flexible to allow the tube to bend with a joint during arthroscopic surgery.

### FIELD OF THE INVENTIONS

The inventions described below relate the field of cannulas for use in surgical procedures and more particularly to a flexible instrument port for use during arthroscopic surgery.

### BACKGROUND OF THE INVENTIONS

In arthroscopic surgery, it is common practice to fill the surgical site with clear saline solution (or other clear, inert fluid) in order to keep the field of view through an arthroscope clear. Since even a small amount of blood will cause the surgical site to become cloudy, means for draining and replenishing the irrigation solution are needed throughout the surgery.

For many arthroscopic surgeries, a rigid cannula is provided into the surgical site and is used to deliver irrigation fluid to the site. A tube is attached between the cannula and an elevated fluid reservoir is provided to deliver the fluid to the cannula. In this case, the rigid cannula is known as an inflow cannula. A second cannula or a suction device for draining fluid from the irrigation site is attached to one port of an arthroscope (or is provided separately). The arthroscope is then introduced into the surgical site. Fluid from the fluid reservoir is introduced into the surgical site by opening a valve on the rigid cannula. Fluid then flows from the reservoir and into the surgical site, thereby irrigating the surgical site. Fluid is removed from the surgical site by activating the suction or by allowing fluid to drain through the second cannula on the arthroscope.

In another procedure, fluid can be introduced through the second cannula on the arthroscope and removed by means of the rigid cannula. In this case, fluid flows from the surgical site, through the rigid cannula, through a flexible plastic tube running from the cannula and to a waste fluid receptacle. In this method, the rigid cannula is referred to as an outflow cannula or as a drainage needle.

The rigid cannula can cause problems during the surgical procedure. During arthroscopic surgery, the surgeon often manipulates the patient's joint in order to obtain a different view of the joint or to accomplish some medical procedure. The arthroscope and rigid cannula often remain in the surgical site while the surgeon manipulates the joint. Manipulating the patient's joint with the rigid cannula in the operating site can cause significant problems. The rigid cannula can bruise or puncture soft tissue near the surgical site. In addition, the flow of fluid through the rigid cannula is frequently blocked by anatomical structures.

Devices and methods that address these problems have been described in Bruce, Flexible Inflow/Outflow Cannula, U.S. Pat. No. 5,527,276 (Jun. 18, 1996). Bruce shows a flexible cannula that is increasingly more flexible towards the distal end of the cannula. When a surgeon manipulates a patient's joint while the Bruce cannula is in the joint, the Bruce cannula will bend as the joint bends. Thus, the Bruce cannula avoids the problems associated with a rigid cannula. However, improvements in the Bruce cannula are possible.

### SUMMARY

The methods and devices described below provide for a cannula and instrument port for use during surgical procedures. The cannula is increasingly more flexible towards the distal end of the cannula. The thickness of the cannula is tapered towards the distal end of the cannula and slots are provided in the distal portion of the cannula to increase the flexibility of the distal portion of the cannula. The slots in the cannula are arrayed in rows, with each row disposed along a portion of the longitudinal length of the cannula. The rows are staggered with respect to each other, such that the rows are longitudinally offset from one another.

During a surgical procedure, an obturator or surgical instrument is inserted into the proximal end of the port and extends through the flexible cannula. Curved instruments can be used with the cannula since the distal end of the cannula will bend to conform to the shape of the instrument.

The instrument port also includes a duckbill valve, flapper valve or trumpet valve disposed in the proximal portion of the instrument port. The valve prevents fluid from flowing past the valve as instruments are inserted into and removed from the cannula. The instrument port is also provided with a fluid port to optionally drain fluid from or introduce fluid into the instrument port.

### BRIEF DESCRIPTION OF THE DRAWINGS

FIG. 1 illustrates a flexible inflow/outflow cannula.

FIG. 2 shows a longitudinal cross section of the flexible cannula taken along the line 2-2 shown in FIG. 1.

FIG. 3 shows a cross section of the flexible cannula taken along the line 3-3 shown in FIG. 2.

FIG. 4 is a perspective view illustrating the flexibility of the cannula shown in FIG. 1.

FIG. 5 is a perspective view of the cannula shown in FIG. 1 with a valve and a surgical instrument disposed through the cannula.

FIG. 6 illustrates a valve for use in the cannula shown in FIG. 1.

FIG. 7 shows an exploded view of an instrument port having a flexible distal cannula.

FIG. 8 illustrates an instrument port having a flexible distal cannula and a bendable surgical clamp disposed within the cannula.

FIG. 9 shows an instrument port having a flexible distal cannula and a proximal V-clamp integrally formed with a rigid tube.

FIGS. 9a, 9b and 9c illustrate alternative structures for the proximal clamp shown in FIG. 9.

FIG. 10 shows an instrument port having a flexible distal cannula and a proximal pinch clamp integrally formed with the rigid tube.

FIG. 11 shows an instrument port configured with another pinch clamp.

FIG. 12 shows an instrument port having a flexible distal cannula, a finger grip and a proximal stop-cock valve.

FIG. 13 shows a cannula having differently sized slots.

FIG. 14 shows a cannula having slots sized and dimensioned to allow fluid to flow into the cannula when the cannula is bent.

FIG. 15 shows a partial longitudinal cross section of a cannula with a non-linearly tapered outer diameter.

### DETAILED DESCRIPTION OF THE INVENTIONS

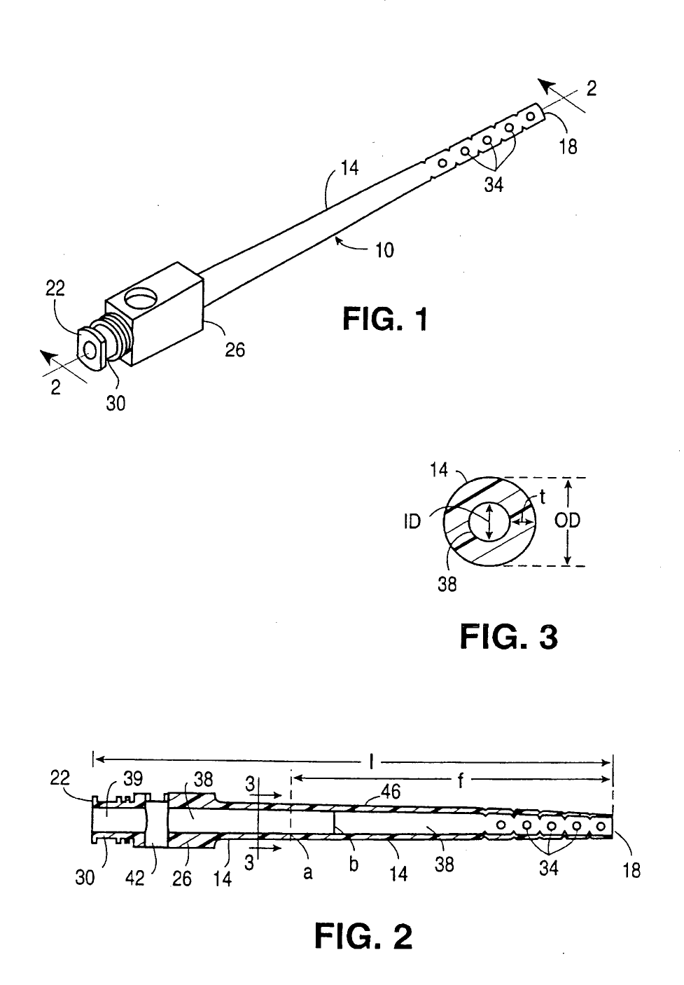

FIG. 1 illustrates a flexible cannula 10 for use in delivering liquids to or from a surgical field during arthroscopic surgery. The cannula 10 comprises a stem 14, a distal end 18, a proximal end 22, a rigid tube 26 and an attachment means 30. A plurality of apertures 34 are positioned near the distal end 18 of the stem 14. The apertures may be provided with rounded edges to make inserting the cannula into an operating site easier.

FIG. 2 illustrates that the cannula 10 is a hollow device having a lumen 38 that extends from the distal end 18 through the stem 14, and into the rigid tube 26. A lumen 39 extends from the proximal end 22, through the attachment means 30, and into the rigid tube 26. The cannula 10 has a total length “1” of approximately four inches.

The lumens 39 and 38 are open at the proximal end 22 and distal end 18, respectively, to provide a means for liquid to enter and exit the cannula 10. Additionally, the apertures, fenestrations or slots 34 extend through the stem 14 and into the lumen 38, thereby providing a plurality of passageways for fluid to flow into or out of the lumen 38.

The stem 14, rigid tube 26 and attachment means 30 are most conveniently manufactured by injection molding from a polymeric material, such as high density polyethylene, to form a single piece instrument. The high density polyethylene sold by Dow Chemical Company under the designation Dow HDPE 25355N Resin (narrow molecular weight distribution copolymer) works quite well. Other materials compatible with use inside the human body and having the requisite flexibility could also be used to manufacture the cannula 10, and other fabrication techniques may be used to manufacture the cannula.

The rigid tube 10 includes an aperture 42 for accepting a valve 44 (shown in FIGS. 5 and 6) that controls the flow of fluid through the cannula 10 by providing a connection between the lumens 38 and 39. The attachment means 30 is a connector, such as a luer lock connector, which allows other pieces of equipment (e.g. fluid supply lines) to be attached to the cannula 10.

The stem 14 is an elongated structure extending from the rigid tube 26 to the distal end 18. FIG. 3 illustrates that a cross section of the stem 14 is circular in shape.

In the cannula 10, the stem 14 is tapered along its length, meaning that the outside diameter of the stem 14 is greater at the proximal end of the stem 14 than it is at the distal end 18, and decreases smoothly from the proximal end of stem 14 to the distal end 18. In other words, the thickness of the cannula wall decreases along the distal length of the cannula. (As shown below, the cannula need not be tapered to retain the property of increasing flexibility towards the distal end of the cannula.)

For example, a cannula designed for arthroscopic surgery of the knee has the following dimensions: The outer diameter of the stem 14 at the reference line “a” is approximately 0.169 inches and the inner diameter at this line is approximately 0.119 inches. The outer diameter of the stem 14 at the distal end 18 is approximately 0.119 inches and the inner diameter at this point is approximately 0.090 inches.

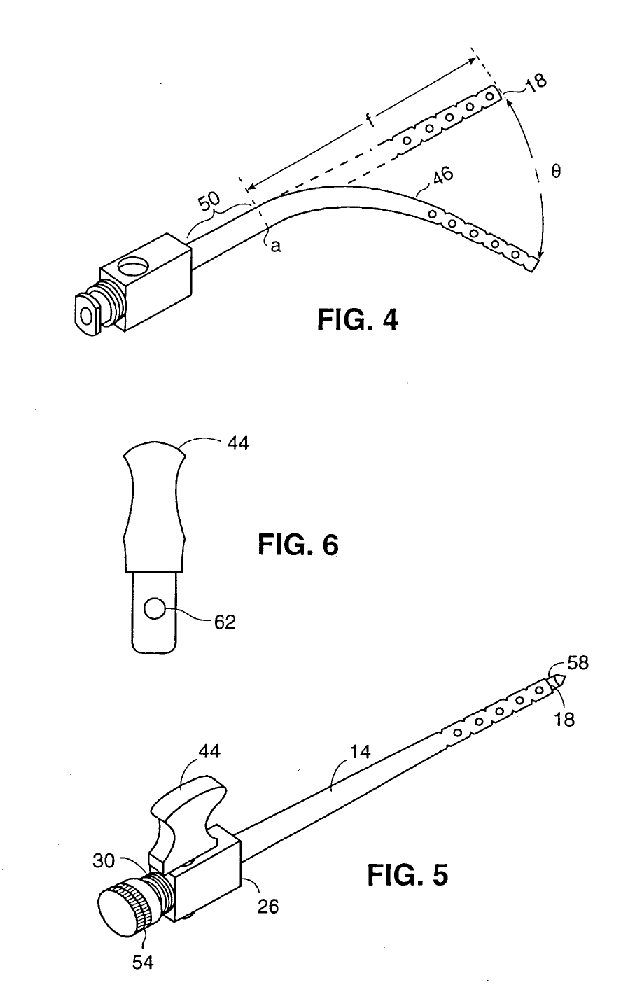

FIG. 4 illustrates that the flexible segment 46 has sufficient flexibility to bend through an angle (θ). The cannula shown in FIG. 4 is thus useful during arthroscopic knee surgery. The thickness “t” of the stem 14 is about 0.025 inches at the reference line “a” and about 0.0145 inches at the distal end 18. This decrease in thickness of the stem 14 over the distance “f” results in the stem 14 becoming progressively more flexible as the distal end 18 is approached from the reference line “a.” The increase in flexibility of the stem 14 is referred to as “progressive flexibility.”

When the distance “f” is about 2.5 inches, the reference line “a” marks the region of the stem 14 where the stem 14 begins to bend when a person grasps the proximal end 22 in one hand and applies downward force to the distal end 18, using the other hand, sufficient to cause the stem 14 to bend through an angle (θ) of approximately ninety degrees. This force is comparable to what would be exerted by the knee while the knee is bent during arthroscopic surgery. Under these conditions, a rigid segment 50 of the stem 14 has sufficient thickness “t” so that it remains inflexible relative to the flexible segment 46. When the downward force is removed, the stem 14 returns to its original “straight” position.

FIG. 5 illustrates the cannula 10 with the valve 44 positioned in the rigid tube 26. A surgical instrument 54 is shown inserted in the lumen 38 of the cannula 10. The tip 58 of the instrument 54 extends outwardly from the distal end 18 of the cannula 10 while the proximal end of the instrument 54 is screwed over the attachment means 30, thereby firmly securing the instrument 54 inside the cannula 10.

FIG. 6 illustrates that the valve 44 includes an aperture 62 that allows liquid to flow through the valve 46 when the valve 44 is open, thereby connecting the lumens 38 and 39. When the valve 46 is rotated such that the valve is closed, liquid can no longer pass through the aperture 62, thereby preventing liquid from flowing between the lumens 38 and 39.

When the knee is bent during a surgical procedure, the flexible segment 46 has sufficient flexibility to bend with the knee inside the compressed suprapatellar pouch. Because the stem 14 bends with knee, the flow of liquid into or out of the distal end 18 is not blocked. Additionally, since the stem 14 is flexible, there is much less likelihood that it will bruise or puncture tissue when the knee is bent.

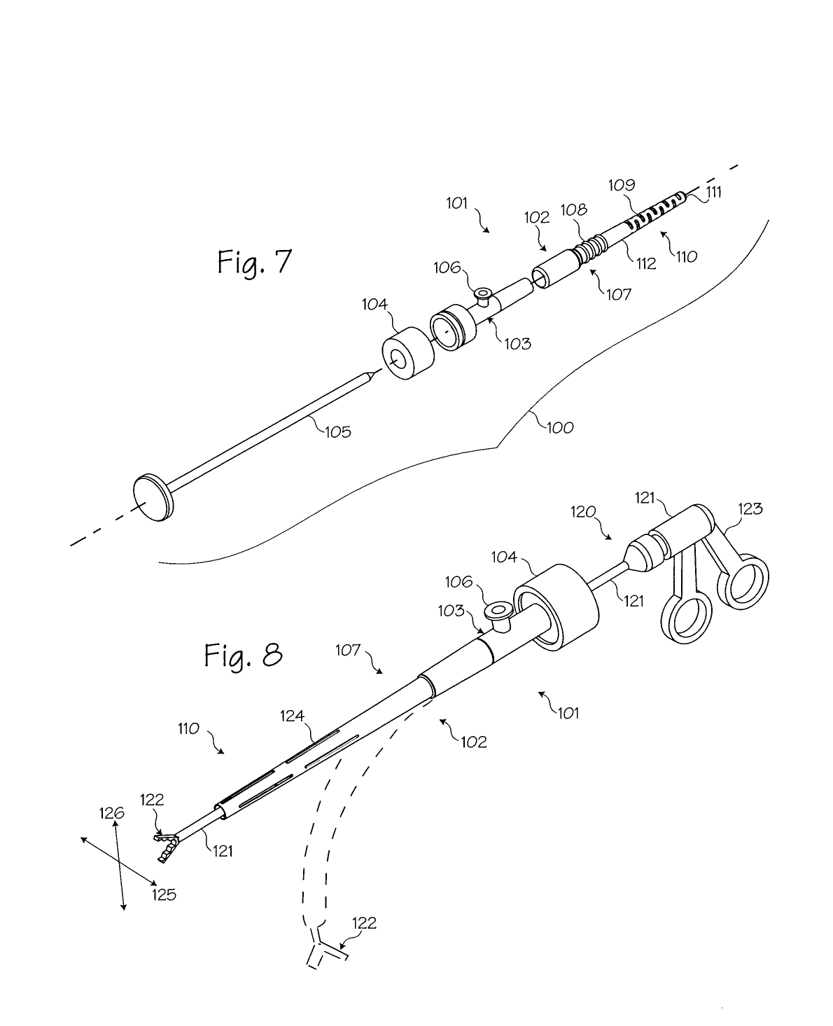

FIG. 7 shows an exploded view 100 of an instrument port 101 having a flexible distal cannula 102. The instrument port is useful for many surgical procedures, including arthroscopic surgical procedures. The instrument port has a cylindrical rigid tube 103, a duckbill valve 104 disposed proximally of the rigid tube and a flexible, tapered cannula 102 disposed distally of the rigid tube. The duckbill valve acts as a check valve which prevents fluids from flowing proximally through the valve while allowing an obturator 105 or other surgical instrument is inserted through the valve, into the rigid tube and through the cannula. The valve closes down upon the instrument and thus prevents substantial flow of fluids proximally through the valve. Thus, instruments may be inserted and removed as needed without removing the flexible cannula from the operating area and without fluids flowing out of the valve. A fluid port or valve fitting 106 is provided in fluid communication with the rigid tube. The fluid port allows a surgeon to introduce or drain fluid to or from the operating site without withdrawing the instrument from the instrument port.

The flexible cannula is removably attached to the rigid tube, though the cannula may be fixedly attached to or integral with the rigid tube (or provided with a unitary construction). The cannula is tapered such that the cannula has increasing flexibility towards the distal end of the cannula. The outer surface of proximal portion 107 of the cannula is also corrugated or provided with ridges 108 to strengthen the cannula and decrease the flexibility of the proximal portion of the cannula. The ridges also help to prevent the cannula or instrument port from being unintentionally forced out of the operating field. The ridges are circumferentially disposed around the cannula (or extend radially from the tube.)

Apertures, fenestrations or slots 109 are disposed in the distal portion 110 of the cannula to allow fluid to flow into or out of the cannula lumen and also to increase the flexibility of the distal portion of the cannula. The slots are circumferentially oriented in the sense that the slots are narrow along the longitudinal length of the cannula, as compared to the circumferential width of the slots. The slots may be provided with rounded edges to decrease the chance that an instrument will snag against the edges of the slots.

The slots are disposed in longitudinal rows, with each row disposed along a longitudinal line of the cannula. The rows are longitudinally staggered with respect to each other in that the rows are longitudinally offset from one another. (The proximal and distal edges of the slots in a particular row do not lie along the same circumferential line as the proximal and distal edges of the slots of the rows disposed to either side of the particular row.) The slots thereby form a zigzag pattern along the outer surface of the cannula, and the slots may be referred to as longitudinally staggered slots. (If the rows are all about parallel to each other and the slots are aligned with each other, then the slots also lie along circumferential lines.)

The longitudinally staggered slots relieve stress on the distal portion of the cannula when the cannula is bent. Thus, the longitudinally staggered slots increase the resistance of the cannula to kinking or collapsing. Kink resistance and collapse resistance may be further increased with braided fiber reinforcements in the flexible cannula. Similarly, the entire cannula may be reinforced with braided fibers. (The fibers increase the ability of a surgeon to transfer torque through the cannula.) Longitudinally staggered slots provide the additional benefits of high flexibility along two axes of bending and create large open arcs for superior fluid flow into and out of the cannula.

The flexible cannula is made of a soft plastic material having a durometer value of about 10 Shore A to about 45 Shore D. The cannula is provided with a blunt distal end 111 that is softer than the shaft 112 of the cannula. This, combined with the flexibility of the cannula, reduces the likelihood of accidental trauma to the patient during a surgical procedure. (The distal end may be sharp for other surgical procedures.) A low friction coating may be provided or applied to the outer surface of the cannula to further increase the ease of insertion into an operating site. For example, the cannula may be provided with a Teflon® (expanded polytetrafluoroethylene or ePTFE) coating or covered with a water-activated lubricant.

The cannula is compatible with radiofrequency surgical probes. The cannula is also thermally resistant so that electrical or thermal instruments may be operated through the cannula. In addition, the cannula may be made of an optically absorbing or scattering material so that laser light does not reflect from the cannula. The plastic cannula is also electrically non-conductive.

FIG. 8 illustrates an instrument port 101 having a flexible distal cannula 102 and a bendable surgical clamp 120 disposed within the cannula. The instrument port includes a rigid tube 103, a flexible cannula 102 attached to the rigid tube, a valve 106 attached to the rigid tube, and a duckbill valve 104 attached to the proximal portion of the rigid tube. The surgical clamp 120 has an instrument housing 121, jaws 122 rotatably attached to the instrument housing and handles 123 for actuating the jaws. (The surgical clamp is shown to illustrate the use of the flexible cannula with a flexible instrument. Other flexible instruments may be used with this instrument port.)

Longitudinally oriented slots 124 in the cannula are disposed in the distal portion 110 of the cannula. (The slots are longitudinally oriented in that the longitudinal length of the slots is greater than the circumferential width of the slots). The slots are longitudinally staggered to increase the kink and collapse resistance of the cannula. The longitudinally oriented slots decrease the probability that a surgical instrument will snag on the edges of a slot. The slots may be provided with rounded edges to further decrease the chance that an instrument will snag against the edges of a slot.

Since the cannula is flexible, the instrument port is capable of accommodating curved, malleable or flexible instruments used during various surgical procedures. The portion shown in phantom shows that the cannula bends with the instrument. The cannula can bend along either axis 125 or 126. However, the cannula can be made to bend preferentially along one of those two axes. For example, the cannula may be made wider along one axis, thus making the cannula preferentially bend in the direction of the other axis. Accordingly, the slots may be sized and dimensioned for preferential bending of the cannula.

In use, a surgeon inserts the surgical instrument into the instrument port, through the duckbill valve, through the rigid tube, and through the cannula. The surgeon then inserts the cannula and the instrument into the operating space. During the surgical procedure, the surgeon may introduce fluids into the operating space through the fluid port. Fluids flow through the fluid port, through the lumen in the rigid tube and cannula, and out of the slots and distal end of the cannula. Similarly, the surgeon can drain fluids from the operating space through the fluid port.

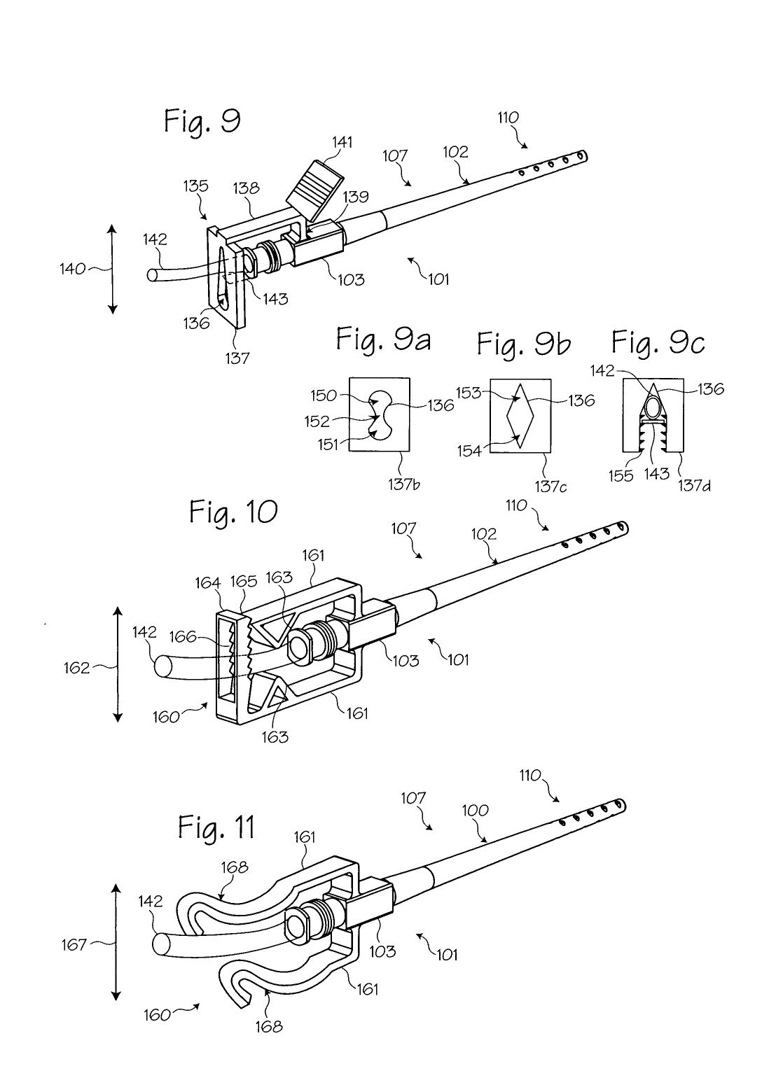

FIG. 9 shows an instrument port 101 having a flexible distal cannula 102 and a proximal V-clamp 135 integrally formed with the rigid tube. The V-clamp, comprising a V-shaped or teardrop shaped opening 136 in a plate 137, is attached to the rigid tube via an “L” brace 138. The connection (shown at area 139) between the rigid tube and the brace is thin enough, or the material flexible enough, that a person may move the brace and plate up and down with respect to the rigid tube, along the direction indicated by arrows 140. A handle 141 may be provided to facilitate movement of the brace relative to a tube 142 or instrument. Optionally, a tongue 143 extending proximally from the rigid tube supports the tube while the clamp clamps down on the tube. Preferably, the brace is made from a durable, resilient plastic or other substance that resists material fatigue while being bent. The brace may be manufactured to resiliently spring back to its originally manufactured position or it may be manufactured to hold a position to which it is bent (as set by the manufacturer).

In use, a tube 142 with flexible walls is secured in fluid communication with the instrument port. The tube is then pressed into the narrow end of the opening 136 in the plate 137. Since the walls of the tube are flexible, the flow of fluid through the tube is restricted (thereby controlling the flow of fluid through the cannula). Fluid flow is restored by pushing the tube into the wide end of the opening in the plate, (by opening the lumen of the tube). In the case of inflexible tubes or solid instruments, the V-clamp can hold the tube or instrument in place with respect to the instrument port.

FIG. 9a shows a second plate 137b that can replace the plate shown in FIG. 9. The opening 136 in the plate is hour-glass shaped, so that fluid flow is unrestricted when the tube is threaded through the first open end 150 or the second open end 151. The tube is pinched closed when pushed into the narrow portion 152 of the opening.

FIG. 9b shows a third plate 137c that can replace the plate shown in FIG. 9. The opening 136 in the plate is diamond-shaped, so that a tube is open to fluid when the tube is threaded through the center of the opening. The tube is pinched closed when pushed into the first narrow portion 153 or the second narrow portion 154 of the opening.

FIG. 9c shows a fourth plate 137d that can replace the plate shown in FIG. 9. A tube 142 is pushed into the V-shaped opening 136 in the plate, thereby pinching the tube and limiting the flow of fluid through the tube. The tongue 143, which can serve as a support for the tube, fits into teeth or ratchets 155 disposed along the edges of the slot. The tongue thus serves as a pawl, thereby securing the position of the tongue with respect to the tube. The tongue also pinches the bottom of the tube as the tongue is ratcheted closer to the V-shaped slot. In addition to the plates shown in FIGS. 9a through 9c, other configurations can be used so long as a tube threaded through the opening in the plate can be moved to pinch the tube closed in one position and leave the tube open to fluid flow in another position.

FIG. 10 shows an instrument port 101 having a flexible distal cannula 102 and a proximal pinch clamp 160. The pinch clamp comprises two “L” braces 161 extending from the rigid tube 103 in the same manner as described in reference to FIG. 9. The braces are flexible with respect to the rigid tube so that the braces may move up and down (along arrows 162) with respect to the rigid tube. Integrally formed with the braces are inwardly extending anvils 163, which in FIG. 10 are inwardly extending hollow wedges. (The anvils may have different shapes and dimensions.) When the braces are pinched together, the anvils pinch the tube which passes between the anvils.

To hold the pinch clamp in a closed position, a ratchet 164 is disposed at the proximal end of one of the braces. The end 165 of the other brace serves as a pawl that fits into the teeth 166 of the ratchet, thereby holding the clamp closed against the force that causes the braces to spring back into their original positions. (The braces are resiliently biased to spring back into their original positions.)

In use, a tube 142 with flexible walls or a rigid instrument is inserted through the clamp and the clamp can serve to hold an instrument in place or control fluid flow through a flexible tube. The braces are pressed together so that the anvils pinch the tube or instrument, thereby restricting the flow of fluid or holding the instrument in place. The end of one of the braces fits into the teeth of the ratchet, thereby holding the pinch clamp in a particular position.

FIG. 11 shows an instrument port 101 configured with another pinch clamp 160. Like the pinch clamp shown in FIG. 10, the braces 161 extend from the rigid tube and are bendable with respect to the rigid tube 103 along the direction of arrows 167. However, in this case the braces are curved so that the proximal portion 168 of the braces are “S” shaped. In use, the two “S” shaped portions of the braces are clamped together and fit snugly together. When clamped together, a tube 142 disposed between the braces is pinched and the flow of fluid through the tube is restricted. Since the braces fit snugly together, the braces remain together until separated.

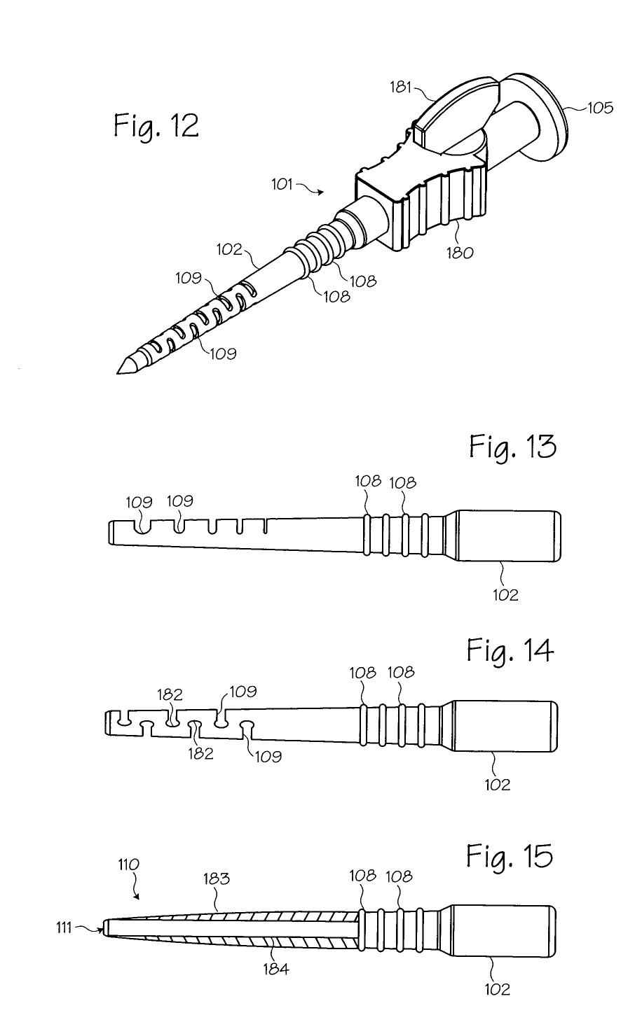

The instrument port may be provided with additional features. For example, FIG. 12 shows an instrument port 101 having a flexible distal cannula 102, a finger grip 180 and a proximal stop-cock valve 181. An obturator 105 or other instrument is disposed through the rigid tube and cannula. The rigid tube is ergonomically shaped into a finger grip so that a surgeon may securely and easily grip the rigid tube and manipulate the instrument port. The stop-cock valve is operable to seal off the flow of fluid to or from the instrument port.

In another example, FIG. 13 shows a cannula 102 having differently sized slots 109. The differently sized slots change how the flexible cannula bends. In the cannula shown in FIG. 13, the slots increase in distal-proximal width towards the distal end 111 of the cannula. In other embodiments the slots may increase in radial depth, or both in width and depth, to accomplish the same effect. In each case, the cannula becomes increasingly more flexible towards the distal end as compared to the flexible cannula shown in FIGS. 1 through 6. In other words, the center of curvature of the cannula (as the cannula is bent from the distal end) is advanced distally with the cannula of FIG. 13 as compared to the cannula of FIGS. 1 through 6. Thus, the way in which the cannula bends may be adjusted by changing the proximal-distal width or the radial depth of the slots.

FIG. 14 shows a cannula 102 having slots 109 that allow fluid to flow into and out of the cannula even if the cannula is bent at an angle. The slots are provided with rounded heads 182 disposed at either end of a given slot. If the cannula is bent at a large angle, and if the rounded heads are not provided at the ends of each slot, then a narrow slot may be pinch closed; thus, fluid will not flow into the cannula. However, the larger openings at either end of each slot will remain open even if a given slot is pinched closed, thereby allowing fluid to flow into the cannula.

The cannula may be made increasingly more flexible towards the distal end by other methods. For example, FIG. 15 shows a partial cross section of a cannula 102 that has an outer diameter which is tapered to create a non-linear shape along the proximal-distal length of the cannula. In this case, the outer diameter of the cannula varies to create a parabolic shape along proximal-distal length of the outer surface of the cannula, as shown in FIG. 15. In other words, the outer surface 183 of the distal portion 110 of the cannula traces a parabolic path from the ridges 108 to the distal end 111 of the cannula. The inside walls 184 of the lumen remain straight and smooth. This cannula may be referred to as a parabolically tapered cannula. The cannula shown in FIG. 15 is not provided with slots, though it is still increasingly flexible along its distal length due to the tapering of the cannula.

The cannula may be differently tapered to form other shapes, so long as the radial thickness of the cannula decreases towards the distal end of the cannula or so long as some other means is provided for making the distal portion of the cannula more flexible than the proximal portion (such as via slots). Another method is to put long, longitudinal grooves (bayonet grooves) along the outer surface of the cannula. Slots may be placed in the grooves to increase the flexibility of the distal portion of the cannula. Slotted grooves also allow fluid to flow into and out of the cannula even if the cannula is pressed against tissue. (The grooves prevent tissue from pressing against the slots and blocking the slots.)

Another method of making the cannula increasingly flexible towards the distal end of the cannula is to provide a number of circumferential grooves that are disposed in the distal portion of the cannula and spaced along the longitudinal length of the cannula. In other words, a number of circumferential sections of the wall of the cannula are provided with a thickness that is less than the thickness of the circumferential wall sections just proximal and just distal of a particular thinner section. For example, one or more circumferential grooves may be placed between any two slots in a particular row of slots (though grooves also may be placed proximally or distally of the rows of slots). Where the rows of slots are about parallel to each other and the slots are longitudinally aligned with each other, the circumferential grooves may extend around the entire circumference of the cannula to further increase the flexibility of the distal end of the cannula.

The instrument port or inflow/outflow cannula may be further modified to meet different surgical needs. For example, the flexible cannula may be fashioned as an integral whole. (The embodiment in FIGS. 7 and 8 shows a removably attached flexible cannula, resulting in a modular device.) In addition, with respect to the valve, valves other than the duckbill valve may be used. Optional valves include a trumpet valve, an elastic ring valve, a flapper valve, a stopcock valve, a clamp valve or other type of valve that seals gas or fluid into a surgical space or allows the passage of a surgical instrument into the surgical space.

In addition, the instrument port's rigid tube and cannula may be provided with different cross sections to change how the cannula bends and to accommodate differently shaped instruments. For example, a cannula having an elliptical cross section is preferentially flexible on its minor axis. This cannula also accommodates instruments having a flat, narrow shape.

The cannula, or one or more of the instrument port components, may be modified to assist medical personnel during a procedure. For example, the cannula or one or more of the instrument port components may be provided with a deflecting mechanism, such as one or more pull wires. Thus, a surgeon will be able to guide the cannula or instrument port more easily during a surgical procedure. To further aid the surgeon, the cannula may be made of a memory material designed to return to its manufactured shape. The cannula may also be made of a material that is malleable. Such a device could be bent into a curved shape.

Yet another cannula or instrument port may be radio opaque so that the surgeon can determine the location of the cannula during a procedure. Similarly, the cannula may have radio opaque markings which indicate a measurement of distance, depth of penetration, degree of deflection or orientation of the device within an operating space. (The radiopaque or radiolucent cannula is particularly useful during hip arthroscopy.) The cannula or instrument port may be transparent so that sutures, specimens or instruments disposed inside the cannula can be visualized within the cannula.

The instrument port V-clamp and pinch clamp may be provided in a variety of shapes and sizes, so long as the clamp is operable to secure an instrument or restrict the flow of fluid within a flexible tube. For example, the “L” brace may be replaced with differently shaped braces and the plate may have different sizes and shapes. The opening in the plate may vary to accommodate differently sized tubes or instruments. Furthermore, the brace and the port may be operably connected by a hinge or a pivot/peg system instead of by a thin section of the brace. In the case of the pinch clamp, the pinch clamp may be secured by a latch, hook or other means for securing the pinch clamp, instead of by the ratchet and pawl system shown in FIG. 10. Similarly, the ratchet, pawl, “L” braces and anvils of the pinch clamp may have different sizes and dimensions, so long as the pinch clamp can secure an instrument or restrict the flow of fluid within a flexible tube. In addition, other types of pinch clamps may be used.

The instrument port or its cannula may be specifically designed to accommodate different instruments. For example, the instrument port may be provided with an adjacent accessory port to accommodate a fiber optic, LED or laser lights source. Furthermore, the cannula may be made of an optically conducting material, whereby the cannula itself acts as a light pipe. Other instruments for which the accessory instrument port may be designed include trocars, obturators, scalpels, clamps, electrodes or other instruments. The instrument port may be designed to accommodate multiple instruments; for example, by providing the instrument port and cannula with multiple lumens. In each case, the instrument port accommodates the features of the particular instrument; for example, the instrument port may be provided with extra ports in the rigid tube to accommodate cables or wires used with a particular instrument. In addition, other clamps may be used to restrict the flow of fluid through a flexible tube or to hold an instrument place. For example, an inline roller clamp on the tube or instrument may be used. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

1. A cannula for directing a liquid to or from a site during arthroscopic surgery, said cannula comprising:
a tube having a lumen extending therethrough;
a plurality of longitudinally staggered rows of slots disposed on the distal portion of the tube, wherein each row of slots comprises a plurality of slots disposed along a longitudinal line of the tube, and wherein each slot is in fluid communication with the lumen of the tube;
said tube being increasingly flexible in the direction of the distal end of the tube.

2. The cannula of claim 1 further comprising a plurality of circumferential ridges, said ridges disposed on the proximal portion of the tube.

3. The cannula of claim 1 wherein the slots are longitudinally oriented.

4. The cannula of claim 1 wherein the slots are circumferentially oriented.

5. The cannula of claim 1 further comprising a plurality of circumferential grooves disposed in the distal portion of the cannula, wherein at least one groove is disposed between two particular slots in a row of slots.

6. The cannula of claim 5 wherein the rows of slots are longitudinally aligned with each other and wherein the at least one groove is disposed around the entire circumference of the tube.

7. The cannula of claim 5 further comprising a plurality of circumferential ridges, said ridges disposed on the proximal portion of the tube.

8. The cannula of claim 5 wherein the slots are longitudinally oriented.

9. The cannula of claim 5 wherein the slots are circumferentially oriented.

10. The cannula of claim 5 wherein the tube is characterized by a thickness, and wherein the thickness of the tube progressively tapers along the direction of the distal end of the tube.

11. A surgical instrument port operable to allow the passage of surgical instruments into and out of a surgical space while restricting the flow of fluid to and from the surgical space, said surgical instrument port comprising:
a rigid tube, said rigid tube having a proximal end and a distal end, said rigid tube having a lumen passing through the rigid tube, said lumen sized and dimensioned to accommodate a surgical instrument;
a valve operably connected to the rigid tube, said valve operable to allow the insertion and removal of the surgical instrument into the lumen and through the valve without allowing a substantial flow of fluid proximally through the valve;
a cannula attached to the distal portion of the rigid tube, said cannula comprising:
a flexible tube, said tube having a lumen extending therethrough, said lumen in fluid communication with the lumen of the rigid tube, wherein the diameter of the lumen is sized and dimensioned to receive a surgical instrument;
a plurality of longitudinally staggered rows of slots disposed on the distal portion of the tube, wherein each row of slots comprises a plurality of slots disposed along a longitudinal line of the tube, and wherein each slot is in fluid communication with the lumen of the tube;
said tube being increasingly flexible in the direction of the distal end of the tube.

12. The instrument port of claim 11 further comprising a plurality of circumferential ridges, said ridges disposed on the proximal portion of the tube.

13. The instrument port of claim 11 wherein the tube of the cannula is characterized by a thickness, and wherein the thickness of the tube progressively tapers along the direction of the distal end of the tube.

14. The instrument port of claim 11 wherein the valve is a duckbill valve.

15. The instrument port of claim 11 wherein the cannula is removably attached to the rigid tube.

16. The instrument port of claim 11 further comprising a fluid port operably attached to the rigid tube and in fluid communication with the lumen of the rigid tube.

17. The instrument port of claim 11 further comprising a clamp operably connected to the rigid tube, said clamp operable to restrict the flow of fluid of a second tube in fluid communication with the instrument port.

18. The instrument port of claim 11 wherein the slots are longitudinally oriented.

19. The instrument port of claim 11 wherein the slots are circumferentially oriented.

20. A system for performing arthroscopic surgery, said system comprising:
a surgical instrument port operable to allow the passage of surgical instruments into and out of a surgical space while preventing the backflow of fluid from the surgical space, said surgical instrument port comprising:
a rigid tube, said rigid tube having a proximal end and a distal end, said rigid tube having a lumen passing through the rigid tube, said rigid tube sized and dimensioned to accommodate a surgical instrument;
a valve operably connected to the rigid tube, said valve operable to allow the insertion and removal of the surgical instrument through the valve without allowing a substantial flow of fluid proximally through the valve;
a cannula attached to the distal portion of the rigid tube, said cannula further comprising:
a flexible tube, said tube having a lumen extending therethrough, said lumen in fluid communication with the lumen of the rigid tube and said tube sized and dimensioned to receive a surgical instrument;
a plurality of longitudinally staggered rows of slots disposed on the distal portion of the tube, wherein each row of slots comprises a plurality of slots disposed along a longitudinal line of the tube, and wherein each slot is in fluid communication with the lumen of the tube;
said tube being increasingly flexible along the direction of the distal end of the tube; and
a surgical instrument extending through the lumen of the rigid tube and through the lumen of the cannula, said surgical instrument operable to perform a surgical procedure.

21. The system of claim 20 wherein the surgical instrument is curved.

22. A method of performing arthroscopic surgery, said method comprising the steps of:
providing a surgical instrument port operable to allow the passage of surgical instruments into and out of a surgical space while preventing the backflow of fluid from the surgical space, said surgical instrument port comprising:
a rigid tube, said rigid tube having a proximal end and a distal end, said rigid tube having a lumen passing through the rigid tube, said lumen sized and dimensioned to accommodate a surgical instrument;
a valve operably connected to the rigid tube, said valve operable to allow the insertion and removal of the surgical instrument through the valve without allowing a substantial of fluid proximally through the valve;
a cannula attached to the distal portion of the rigid tube, said cannula further comprising:
a flexible tube, said tube having a lumen extending therethrough, said lumen in fluid communication with the lumen of the rigid tube, wherein the diameter of the lumen is sized and dimensioned to receive a surgical instrument;
a plurality of longitudinally staggered rows of slots disposed on the distal portion of the tube, wherein each row of slots comprises a plurality of slots disposed along a longitudinal line of the tube, and wherein each slot is in fluid communication with the lumen of the tube;
said tube being increasingly flexible in the direction of the distal end of the tube; and
providing a surgical instrument suitable for performing an arthroscopic surgery procedure;
inserting the surgical instrument through the lumen in the port, through the valve and through the cannula;
inserting the surgical instrument and cannula into an operating space; and
performing the arthroscopic surgery procedure.
